(19) United States
(12) Patent Application Publication
(10) Pub. No.: US 2016/0015962 A1
(43) Pub. Date: Jan. 21, 2016

(54) Smart Patch For Wound Management

(71) Applicants: Mehdi Shokoueinejad Maragheh, Madison, WI (US); Sarah R. Sandock, Milwaukee, WI (US); Akshay Kumar, Madison, WI (US); Yei Hwan Jung, Madison, WI (US)

(72) Inventors: Mehdi Shokoueinejad Maragheh, Madison, WI (US); Sarah R. Sandock, Milwaukee, WI (US); Akshay Kumar, Madison, WI (US); Yei Hwan Jung, Madison, WI (US)

(21) Appl. No.: 14/801,134

(22) Filed: Jul. 16, 2015

Related U.S. Application Data
(60) Provisional application No. 62/025,269, filed on Jul. 16, 2014.

(51) Int. Cl. A61N 1/04; A61N 5/06; A61F 13/00

(52) U.S. Cl. CPC A61N 1/0468; A61F 13/00055; A61N 5/0625; A61F 13/00021; A61F 13/00034; A61N 1/0492; A61N 1/0404; A61N 2005/0645; A61N 2005/0659; A61N 2005/0661; A61N 2005/0662

(57) ABSTRACT

A flexible patch is provided that is capable of emitting light in the UV, visible, and/or infrared electromagnetic spectrums. The patch contains a feedback process and system using one or more sensors and a controller on the patch to (1) accelerate the wound healing process by providing adaptable, controlled light exposure and electrical stimulation, (2) monitor the healing process for signs of infection (3) eliminate bacterial infections by sanitizing the infected site and (4) relaying the information wirelessly to a central location for storage and interpretation by a physician as well as by providing the ability to receive feedback and operating instructions from the physician from a remote location.

### CROSS-REFERENCE TO RELATED APPLICATION

This application claims priority from U.S. Provisional Patent Application Ser. No. 62/025,269, filed on Jul. 16, 2014, the entirety of which is expressly incorporated by reference herein.

### FIELD OF THE INVENTION

The invention is directed generally to phototherapy, and more particularly, to devices for administering wound sensing using sensors and wound healing using radiation and electrical stimulation to a targeted site on a patient.

### BACKGROUND OF THE INVENTION

Phototherapy is the therapeutic use of light. It is an effective method of treating wounds and reducing pain in humans. External phototherapy has been effective in treating various medical conditions, such as, but not limited to, bulimia nervosa, herpes, psoriasis, seasonal affective disorder, sleep disorders, acne, skin cancer, and other conditions. Phototherapy is typically administered to a patient using a light source that is formed of either a bank of lights or a fiber optic light source. Typically, the light sources used in phototherapy are fluorescent tubes, metal halide lamps, or light-emitting diodes (LEDs).

While light sources formed as banks of lights are still being used, they have several disadvantages. For instance, using light banks requires that patients wear uncomfortable eye protection. These devices also require that patients remain relatively stationary while receiving treatment. Furthermore, these devices are typically large and immobile. Therefore, patients must visit specific locations, such as hospitals, each time a dosage is needed.

Fiber optic light sources were developed as a substitute for phototherapy devices containing light banks but they too have drawbacks. For instance, fiber optic lights typically deliver lower overall amounts of light than the light banks, thereby reducing the effectiveness of the therapy. Additionally, fiber optic lights are often used in conjunction with fiber optic mats having specific geometries. Often times, the fiber optic mats are compromised when they are forced into contact with patients skin surfaces. This undesirably results in unequal concentration of light intensity, with a greater light intensity near the light source than at other portions of the fiber optic mat.

Thus, a need exists for a phototherapy device that delivers light in a more efficient, flexible, and portable manner.

### BRIEF SUMMARY OF THE INVENTION

According to one aspect of an exemplary embodiment of the invention, a flexible patch is provided that is capable of emitting light in the UV, visible, and infrared electromagnetic spectrums and applying electric impulses through an electrode. Some exemplary embodiments of the present mention also contain a feedback process and system using one or more sensors and a controller on the patch to (1) accelerate the wound healing process by providing adaptable, controlled light exposure and electrical stimulation, (2) monitor the healing process for signs of infection, and (3) eliminate bacterial infections by sanitizing the infected site and (4) relaying the information wirelessly to a central location for storage and interpretation by a physician and to enable the physician to control the opera. The entire system is packaged in an ultra-thin flexible patch that can wrap around the epidermis in a conformal manner to deliver light therapy precisely to a small wound and to allow dynamic monitoring of the wound healing process.

Additional features, advantages and aspects of the invention will be made apparent from the following detailed description taken together with the drawing figures.

### DESCRIPTION OF THE FIGURES

The drawing figures illustrate the best mode currently contemplated of practicing exemplary embodiments of the present invention.

FIG. 1 is a perspective view of one exemplary embodiment of a patch constructed according to the present invention.

FIG. 2 is an exploded schematic view of the various layers present in another exemplary embodiment of the patch of the present invention.

FIG. 3 is a schematic view of an exemplary embodiment of a control circuit for use with the patch of the present invention.

FIG. 4 is a schematic view of an exemplary embodiment of the data transmission operation of the patch of the present invention.

### DETAILED DESCRIPTION OF THE INVENTION

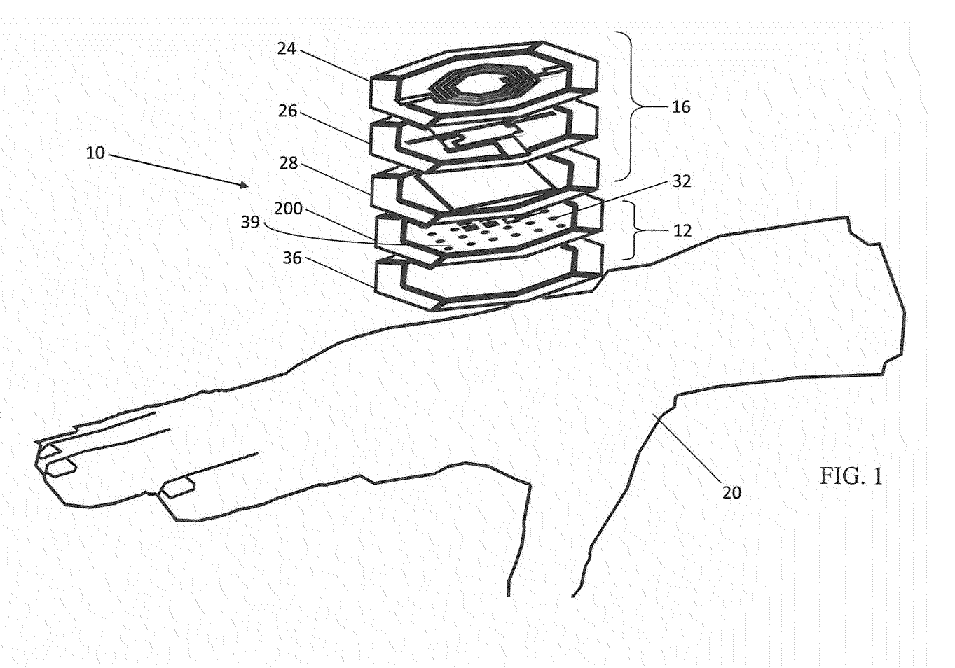

With reference now to the drawing figures in which like reference numerals designate like parts throughout the disclosure, one exemplary embodiment of a flexible patch constructed according to the present invention is illustrated generally at 10 in FIG. 1. The patch 10 is capable of being attached to any mammalian tissue 20, such as human or animal epithelial tissue, among others and functions by emitting light in the UV, visible, and infrared electromagnetic spectrums. Some exemplary embodiments of the patch 10 of the present invention also contain a feedback process using one or more sensors and a controller to (1) accelerate the wound healing process by providing adaptable, controlled light exposure and electrical stimulation, (2) monitor the healing process for signs of infection (3) eliminate bacterial infections by sanitizing the infected site and (4) relaying the information wirelessly to a central location for storage and interpretation by a physician.

Vital wound-site data from sensors and treatment data, including the dosing schedules, are stored with timestamps for real-time analysis by physicians. This enables physicians to make decisions and adjustments to the treatment remotely thereby making the patch 10 a valuable tele-therapeutic wound care device.

In some exemplary embodiments, all of the components of the patch 10 exist as a single system. That system may be reusable or disposable. In other embodiments, the patch 10 is the combination of multiple systems that are each either re-usable or disposable depending on the particular configuration of the patch 10, such that various embodiments of the patch 10 can include combinable components that function together to provide the benefits of the patch 10.

For example, in one exemplary embodiment, the photodynamic therapy patch 10 comprises a flexible body 11 having two independently flexible components that each can conform to the shape of the tissue to which the patch 10 is applied. The first system is a disposable patch, layer, module or portion 12. The disposable patch 12 may house the LED(s) 32, either singularly or in an array, and the cover 22. In various exemplary embodiments, the disposable patch 12 may be in different sizes and shapes, and may be clear or transparent or any color. In one embodiment, the LED(s) 32 may be mounted on the disposable patch 12, or in another embodiment, they may be mounted on the second system, the reusable patch, module or portion 16. In one exemplary embodiment, the reusable module 16 includes layers of flexible, thin film electronics 18 (such as antennas, the controller, non-volatile memory, a battery, sensors, electrical stimulation system and microscale ultra-thin LED arrays), all in an ultrathin format so that they can be wrapped around or attached to the skin or tissue 20 in which the wound is present. Alternatively, the reusable layer, module or portion 16 encloses all the components that do not make contact with the tissue or skin. This includes the battery 26, controller 28, wireless communication system and its associated antenna 24. In this alternative embodiment, the disposable layer, module or portion 12 encloses all the components that make contact with the tissue or skin, includes all LEDs 32, electrical stimulation electrodes 39 and sensors 30.

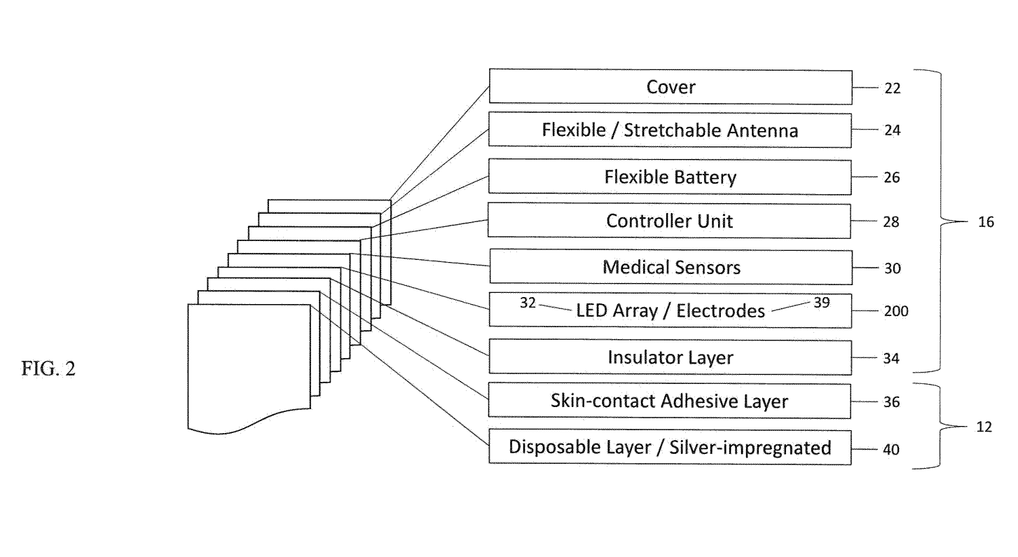

Looking now at FIG. 2, an exemplary diagram of the layout of the various components that make up one exemplary embodiment of the patch 10. The exemplary embodiment illustrating the reusable module 16 is comprised of an exterior protective cover 22 a stretchable, flexible antenna 24, a flexible battery 26, a controller unit 28, medical sensors 30, a electronic stimulation system 200 which can include a light source, such as and LED(s) array 32 and/or an electrode 39, an insulator layer 34, and a skin contact layer 36, with a removable disposable layer 40.

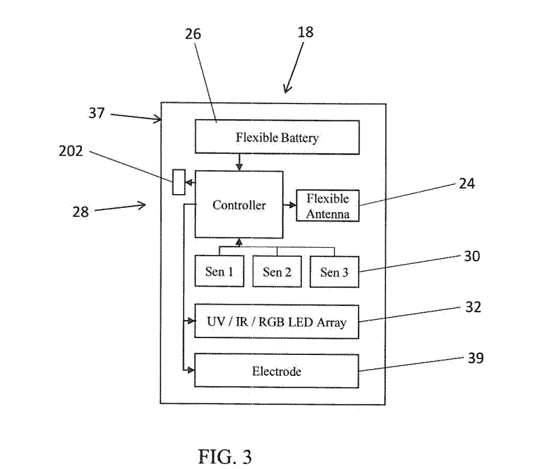

In this exemplary embodiment, the stretchable antenna 24 enables data communication Using the antenna 24, the electronic circuit(s) 36 (FIG. 3) is configured to either be operably connected to or wirelessly transmit all of the data gathered with time-stamping from the various sensors 30 to an external monitor or device 102,104 via a suitable network 100. This may be done via Bluetooth, NFC, Wi-Fi, or any other suitable means of wireless data transmission. This allows patients and/or doctors to continuously monitor the conditions of wound and the progress of the therapy, such that the dosing and timing provided by the patch 10 can be modified by clinicians through communications with the patch 10. Physicians are able to change the treatment regimen and dosage as needed by communicating with the patch 10 through the antenna 24.

The battery 26 in an exemplary embodiment may be flexible, rechargeable and/or disposable and use zinc and manganese dioxide battery chemistry and can include multiple flexible batteries connected together (in series or parallel) configurations to achieve the required power consumption levels for the patch 10, The battery 26 and the controller 28 can also be connected to suitable power regulation circuitry (not shown) disposed on circuit 37 that is capable of regulating the power from the battery 26 before providing it to the controller 28, sensors 30, LEDs 32 and wireless communication system/antenna 24 and is capable of monitoring the battery levels and providing battery health to the controller 28.

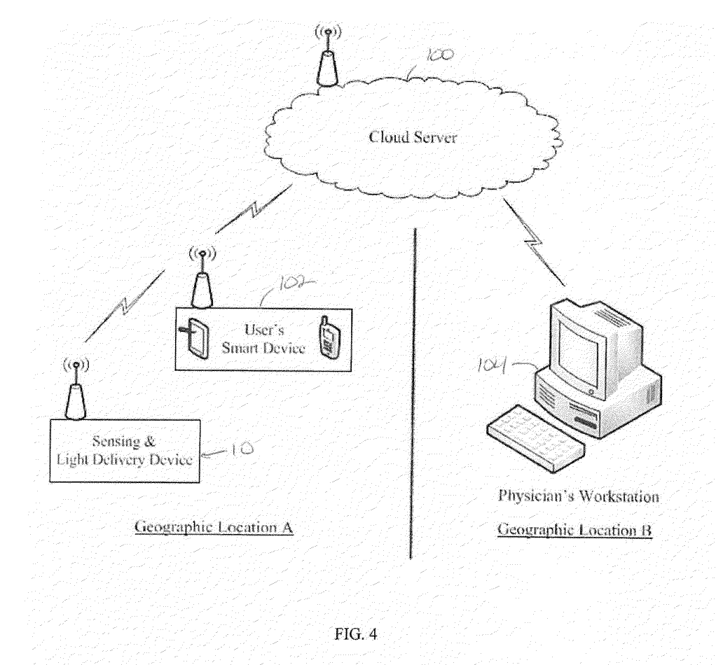

In an exemplary embodiment, the controller unit 28, as shown in FIG. 3 is a physical controller chip 38 disposed in the electronic circuit 37 and running firmware capable of closed-loop feedback in a suitable manner. In an exemplary embodiment, the controller chip 38 interacts with the various sensors 30, and transfers information with an external monitoring system, such as a PC 104, smartphone 102, or other electronic device, as shown in FIG. 4.

In an exemplary embodiment shown in FIG. 4, the external monitoring system may be a cloud server 100 that feeds information to a user's smart device 102 and physician's workstation 104. The cloud server 100 will have the capability to store the sensor information without the patient's details. The physician, who has access to the patient, wound data, can then interpret the results using a custom software application to identify the wound healing process and develop quantitative estimates. Based on these estimates, the physician can directly adjust the exposure of lights and/or electrical impulses from the patch 10 on the wound to continue with the healing process.

Further, with the use of the wireless communication system/antenna 24, the controller 28 is operably connected to the wireless communication system 24 to enable transmission of time-stamped sensor data from the sensors 30 through the controller 28 via a suitable network 100 to a remote device 102,104 as well as to enables reception of light source and electrical stimulation commands by the controller 28 from a remote device 102,104 via the network 100. In addition, the controller chip 38 within the controller 28 can configured to execute software instructions stored in a suitable electronic storage medium or database 202 connected to the controller chip 38 to accomplish the functions. The program instructions will be stored in an on-chip or external flash memory. The program execution can be done in any suitable manner, such as by an on-chip RAM (not shown) or external RAM (not shown) so as to conserve operating power.

These adjustment procedures will have redundant safety features so that the physician/patient cannot adjust the treatment course by mistake. The manner in which data is transferred between digital devices in this embodiment will abide by the rules set up by the governing bodies. In an exemplary embodiment, the collected data may be used for big data applications such as (but not limited to) trend predictions, wound healing patterns over a geographic region etc.

In an exemplary embodiment, the controller 38 is capable of producing different types of electronic signals, depending on the requirements of the LEDs 32. The electronic signals could be either analog signals or digital signals with pulse-width modulation. In an exemplary embodiment, the controller 38 also monitors the charge level in the battery 26 and saves data to on-chip non-volatile memory on or separate from the controller 38 to prevent data loss.

The firmware running on the controller 38 may be bare-metal or it may have an operating system depending on the battery capacity and the power consumption of the controller 38. In an exemplary embodiment, by analyzing the information obtained from the sensors 30 through the close-loop feedback system, such as PH, moisture, temperature, redness of wound, skin conductivity, amount of fluid present, and combinations thereof among others, the patch 10 is also able to sense infection and alert the patient or physician, as well as change the light dosages from the LEDs 32 to treat the infection utilizing the controller 38.

The patch 10 may accommodate one or more of various medical sensors 30 to monitor the wound status. These medical sensors 30 may be active or passive sensors that are, but are not limited to, those that are:
a) capable of measuring the moisture around the area where is it present using mesh like capacitance sensor array. This measurement utilizes the scatterfield effect. A quantitative measure can be determined for skin moisture as a quantity of water content;
b) capable of sensing and measuring tissue impedance by using mesh like sensor array. These sensors are capable of applying high frequency current in the order of microamperes and capable of reading voltage in order to find the impedance of the tissue;
c) capable of measuring the redness using the micro scale light emitter and photodiode sensor. By analyzing the received light attenuation, the system is capable to measure the ratio of oxygenated (healthy skin) and deoxygenated (dead skin) wound area; and/or
d) capable of measuring the temperature on the area where is it present using a semiconductor-based stand-alone temperature sensing chip.

The types of active sensors that are in direct or very close contact with the wound covered by the patch 10 and may be embedded in the patch or patch 10 include, but are not limited to, pH sensors such as silicon based ion sensitive field effect transistors to monitor pH, moisture sensors, and biosensors to detect the presence of bacteria. There are several ways to detect the presence of bacteria using a biosensor, including through facilitative, attenuated, or direct sensing methods using any one of the following: electrical, optical, mechanical, mass, acoustic, thermal, chemical, and magnetic properties. This sensors 30 utilized in the present invention include, but are not limited to the use of any one of those means for the detection of the presence of bacteria. The passive sensors that may be embedded in the patch or patch 10 include, but are not limited to, a photo sensor, such as those employing a silicon-based photodiode, to monitor the light emission of the LEDs 32 to control the redness of the wound, or a temperature sensor, such as those employing a platinum electrode, to monitor the heat generated by the LEDs 32.

The power supply circuitry 37 can include a low-dropout regulator (not shown) to provide a regulated voltage to the controller 38 and other components. Also, the controller circuit 37 can include an 10 expander (or a latch) to accommodate the 10 requirements of the LEDs 32. In one exemplary embodiment the controller microchip 38 is selected from an 8-bit, 16-bit, or 32-bit processor and capable of running bare-metal or an operating system within itself. Additionally, the exemplary embodiment of the controller chip 38 has an electronic data interface to the LEDs 32, sensors 30 and electrode(s) 39 is selected from parallel GPIOs, serial SPI, serial I2C interfaces. If any of the serial interfaces are used, a compatible 10 expander will be used, as discussed previously. Also, in another exemplary embodiment the controller chip 38 is capable of backing up (or storing) critical time-stamped sensor, LED, electrode data in case of a power failure in a non-volatile storage (not shown) for later information retrieval.

In an exemplary embodiment, the electrical stimulation system 200 uses an electrode system 39 in conjunction with or as an alternative to the LEDs 32 to provide and generate a current flow in order to spread throughout the wound site. Following tissue damage, a small injury is generated in order to trigger biological repair. There are many ways to permeate an electrical current flow throughout wound site using electrodes 39, including acupuncture needles, adhesive electrode patch, Multi-layer combination of an electric stimulation with wound dressing with or without the presence of saline. This present invention is not limited to the use of any one of those means for providing pulse electrical stimulation. Electrical stimulation, such as through the use of a mesh electrode 39 as controlled by the controller 28, affects the biological phases of wound healing in the inflammation phase, the proliferation phase, and the epithelialization phase to speed healing of the wound.

In an exemplary embodiment, the various layers 22-36 and 40 housing the various sensors 30 and circuits 37 are made using sheets of suitable plastic materials that are inert, such as polyethylene glycol or parylene, which can be clear and/or transparent, and/or certain plastic electronics technology where the active electronics are fabricated on a thin sheet of plastic, such as polyirnides, for example.

In a further exemplary embodiment, the LED(s) array 32 uses micro-scale, ultrathin light emitting diodes that can accurately target small or large wounds. Because the LEDs 32 are ultrathin and small in area, they will not be affected by the bending of the patch 10 because the spacing between LEDs 32 allow for mechanical stress relaxation. The spatial distribution of the micro LEDs also manages heat generated by individual LEDs and allows low temperature light therapy, In an exemplary embodiment of this invention, the LEDs 32 are efficient, inorganic LEDs. However, this is not a requirement of the invention and other embodiments may use organic LEDs (OLEDs), among others. In some embodiments, inorganic and organic LEDs 32 may be used together. Another advantage of the small area of each LED is that the patch as a whole generates less heat, while maintaining the same light extraction.

The LED(s) array 32 allows for the production of light in various wavelengths. In the preferred embodiment, a single patch 10 is capable of limiting the more harmful UV exposure, as well as limiting chances of UV immunity or resistance by selectively emitting light in the UV-A (wound healing acceleration), UV-B (wound healing acceleration), UVC (sanitization and germicidal purposes), visible light (other healing acceleration), and infrared (vasodilation and wound healing acceleration) spectrums. Any color in the visible light spectrum is produced by combining red, green, and blue (ROB) wavelengths to accelerate the wound healing process by using blue light. For example, the LEDs 32 of the patch 10 reduce exposure to UV-C. UV-C is the deepest wavelength of the UV spectrum which has the most mutagenic.: properties. UV-C is only used if infection is detected or a does is explicitly needed (1). In particular, in one exemplary embodiment, the patch 10 uses UV-A and UV-B LEDs 32 to facilitate the wound healing process, and a 25% reduction in healing time (2) along with greater induction of inflammatory response and wound healing growth factors. Further, infrared (IR) light to facilitate the wound healing process by inducing vasodilation and inducing wound healing growth factors. (3) In an exemplary embodiment, the LEDs 32 are arranged so that the patient receives a consistent dosage across the entire target area.

Other potential attributes of the LEDs 32 that can be utilized in the patch 10 include, but are not limited to:
1. Each LEDs 32 will be ultrathin (7˜10 μm), micro scale (100 μm×100 μm), so that it will not be affected by the bending of the patch 10.
2. For larger area, these microscale LEDs 32 would be assembled in a deterministic format in arrays.
3. Compared to having a single large area LED 32, this would generate lower heat, but have same light extraction.
4. For example, a 500 μm×500 μm single LED will have generate more heat than by putting 100 μm×100 μm LEDs in a 5×5 array form with some spacings in between.(4)
5. In terms of flexibility this can provide benefits because the spacings between the LED array will allow mechanically stress relaxation as compared with a single larger LED that will break when bent.
6. For different colors of LEDs, we can use different types of inorganic compounds, and combining RGB diodes in the LEDs 32 can cover the entire visible light spectrum.
7. LEDs will be pulse operated to save energy and dissipate less heat. It is experimentally proved that at 10 Hz operation, there is only 0.5 C temperature increase, e.g., 36 C to 36.5 C. (5)

Furthermore, the LEDs 32 may, in some embodiments, be insulated by an insulating layer 34, such as a coating of PDMS or high melting point transparent polymers, thereby limiting the amount of heat transferred to the skin 20. The insulating layer 34 also provides electrical insulation between the electronics above in the patch 10 and the skin 20, as well as helping dissipate heat generated by the LEDs 32.

In another exemplary embodiment, the bottom or skin contact layer 36 comprises a biocompatible rubber adhesive that has semi-permanent reusability on the surface contacting the patient's skin 20. This layer 36 may be transparent, and it is porous to allow for heat and vapor dissipation, as well as to allow the sensors 30 to receive the necessary information from the patient's body. In another exemplary embodiment, this layer 36 does not contain an adhesive layer and serves only as a barrier between the components of the patch 10 and the skin 20 of the patient. In other embodiments, the cover 36 may be larger than the remainder of the patch 10 and contain an adhesive on the portion of the layer 36 that extends beyond the patch 10 and contacts the patient's skin 20.

In another exemplary embodiment, the disposable section or layer 40 may be embedded with additive therapies such as embedded silver or antibiotic gels, to further aid and accelerate the healing process. The layers 36,40 of the device that contact the skin may also be coated with bio-inert materials, such as PEG, to prevent bacterial attachment to the device 10 and sensors 30. Further, the layers 36,40 can be combined into a single layer in additional exemplary embodiments.

The cover 22 of the device 10, as shown in FIGS. 1 and 2, may have color or color-metric indicators (not shown) to communicate information to the user. For example, in an exemplary embodiment the device 10 has a safety indicator (not shown) that illuminates when UV radiation is being used, an infection indicator (not shown) that illuminates when bacteria have been sensed in excess of a threshold limit, or an indicator that illuminates when there is a technical problem with the patch. These indicators are made either with a hi-stable display such as the electrophoretic method behind electronic ink seen in such brands as e-ink, or with LEDs.

Further, the benefits of the patch 10 of the exemplary embodiments of the present invention include, but are not limited to:
an increase in efficiency of LEDs by using inorganic LEDs which have 10-20% light efficiency over the 2% efficiency of organic LEDs. This reduces both the power needed to reach the same energy (1-80 J/cm²) delivered.
a controller to provide signals to the LEDs so as to reduce heat buildup.
wireless communication to a smartphone or similar devices.
a rechargeable battery.
feedback to provide controlled UV exposure (utilizing a broad spectrum of LEDs)
medical sensors that can monitor the wound status (sensors measuring PH, temperature, moisture, and redness of the skin)

### REFERENCES CITED

The following references have been cited in the specification and are expressly incorporated by reference herein in their entirety:
(1) Gupta et al. 2013. Ultraviolet Radiation in Wound Care: Sterilization and stimulation. Advances in wound care. 2 (8); 422-437.
(2) Wills E E, Anderson T W, Beattie B L, and Scott A: A randomized placebo-controlled trial of ultraviolet light in the treatment of superficial pressure sores. J Am Geriatr Soc 1983; 31: 131.
(3) Whelen et. al. 2001. Effect of NASA light-emmitting diode irradiation on wound healing. J Clin Laser Ivied Surg. 19(6): 305-14.
(4) Kim and Jung et. al. 2012. High-Efficiency, Microscale GaN Light-Emitting Diodes and Their Thermal Properties on Unusual Substrates. Small, 8 (11): 1643-1649.
(5) Kim et al. 2013. Injectable, Cellular-Scale Optoelectronics with Applications for Wireless Optogenetics. Science. 340: 211.

The many features and advantages of the present invention are apparent from the written description. Further, since numerous modifications and changes will readily occur to those skilled in the art, the invention should not be limited to the exact construction and operation as illustrated and described. Hence, all suitable modifications and equivalents may be resorted to as falling within the scope of the invention. Further, the various aspects, features, embodiments, or implementations of the invention described above can be used alone or in various combinations.

We claim:

1. A patch for management of a wound present in mammalian tissue, the patch comprising:
a. a body adapted to be placed on the tissue over the wound;
b. at least one sensor disposed on the body for the patch.
c. at least one electrical stimulation system on the body for the patch.
d. a controller disposed on the body and operably connected to the at least one electrical stimulation system and the at least one sensor to control the operation of the at least one electrical stimulation system in response to data received from the at least one sensor.

2. The patch of claim 1 wherein the at least one electrical stimulation system is at least one light source.

3. The patch of claim 2 wherein the at least one light source is configured to emit light in at least one of the ultraviolet, infrared or visible light spectrums.

4. The patch of claim 2 wherein the at least one light source is an LED.

5. The patch of claim 4 wherein the at least one light source is an array of LEDs.

6. The patch of claim 1 wherein the at least one electrical stimulation system is at least one electrode.

7. The patch of claim 1 further comprising a wireless communication system operably connected to the controller.

8. The patch of claim 7 wherein the wireless communication system is configured to send wireless signals from the patch representing data obtained from the at least one sensor to a remote device.

9. The patch of claim 7 wherein the wireless communication system is configured to receive wireless signals from a remote device for use by the controller in operating the at least one electrical stimulation system.

10. The patch of claim 1 wherein the at least one sensor is configure to sense moisture, tissue impedance, redness, temperature, or combinations thereof.

11. The patch of claim 1 wherein the body comprises:
a. a first module adapted to contact the tissue; and
b. a second module operably connected to the first module.

12. The patch of claim 11 wherein the second module is releasably connectable to the first module.

13. The patch of claim 11 wherein the first module is disposable.

14. The patch of claim 13 wherein the first module includes the at least one electrical stimulation system and the at least one sensor.

15. The patch of claim 13 wherein the second module includes the controller.

16. A method for treating a wound comprising the steps of:
a. providing the patch of claim 1;
b. placing the patch over the wound in the tissue; and
c. operating the at least one electrical stimulation system to treat the wound.

17. The method of claim 16 wherein the patch includes a wireless communication system operably connected to the controller, and further comprising the step of:
a. sensing a condition of the wound using the at least one sensor after operating the at least one electrical stimulation system;
b. transmitting data from the at least one sensor to a remote device using the wireless communication system;
c. receiving data from the remote device; and
d. operating the at least one electrical stimulation system in response to the data received from the remote device.

18. The method of claim 17 wherein the step of receiving data from the remote device comprises receiving operating instructions from the remote device for use by the controller in operating the at least one electrical stimulation system.

19. The method of claim 16 wherein the body includes a first module and a second module; and wherein the step of providing the patch comprises the steps of:
a. placing the first module over the wound in the tissue; and
b. operably connecting the second module to the first module.

20. The method of claim 19 wherein the method further comprises the steps of:
a. disconnecting the second module from the first module after operating the at least one electrical stimulation system to treat the wound; and
b. disposing of the first module.
